(19) United States
(12) Patent Application Publication
(10) Pub. No.: US 2008/0177211 A1
(43) Pub. Date: Jul. 24, 2008

(54) Machine and Method for Vertical Human Stretching

(76) Inventor: John P. Boren, Katy, TX (US)

Correspondence Address: ANDREWS & KURTH, L.L.P., 600 TRAVIS, SUITE 4200, HOUSTON, TX, 77002, US

(21) Appl. No.: 12/013,316

(22) Filed: Jan. 11, 2008

Related U.S. Application Data
(60) Provisional application No. 60/880,161, filed on Jan. 12, 2007.

(51) Int. Cl. A61F 5/00; A63B 26/00; A63B 24/00

(52) U.S. Cl. 602/32; 482/144; 482/4

(57) ABSTRACT

A machine and method of vertically stretching and exercising the human body, specifically the lower half of the human frame. The machine comprises a bed, table, or platform having a user-controlled actuator which raises one end of the bed, table, or platform up to forty degrees above a completely horizontal position such that the user's upper body is raised above the user's lower body. As a consequence, the user's lower body is either partially or fully suspended from the user's point of attachment to the bed, table, or platform. Because the user is in control of the precise actuator movements, and hence the degree of vertical positioning, the machine is much easier and safer to operate and more effective than other exercise or physical strengthening devices.

### CROSS REFERENCE TO RELATED APPLICATION

This application is based upon provisional application 60/880,161, filed on Jan. 12, 2007, the priority of which is claimed.

### BACKGROUND OF THE INVENTION

1. Field of the Invention

This invention relates to a device or machine for stretching and exercising the human body. Specifically, the invention relates to a device or machine for stretching and exercising the lower half of the human body through vertical suspension.

2. Description of the Prior Art

Prior art devices for stretching the human frame have included numerous medical and non-medical mechanical devices. These mechanical devices include both motorized and non-motorized movement, and employ weight stacks, plyometric resistance, and/or gravity to accomplish the goal of stretching, flexing, or unloading the human frame. Purely medical devices have included over-the-door and power-controlled traction devices, among others, which are arranged and designed to decompress the spine. Non-medical devices have typically included gym-type stretching equipment including, but not limited to, gravity balls and Pilates machines, which are arranged and designed to improve flexibility and range of joint motion. However, Pilates equipment, MedX exercise equipment, and other fitness-oriented exercise and stretching units are often uncomfortable, cumbersome, and/or difficult to operate.

Among the more common types of prior art devices for stretching the human frame are “inversion” devices which operate by inverting the human body. These inversion devices suspend the user in an upside down position by the ankles or knees so that the spine and associated structures that produce vertical pressure while upright can be unloaded and stretched. The principle behind these inversion devices is that, by turning the human frame upside down, gravity acting on the human frame in the reverse direction will assist in relaxing the spine and associated structures thereby giving the user a sense of relaxation and well being. However, a major drawback of these inversion devices and their method of operation is that the user's cardiovascular system must operate in an abnormal state, thus potentially causing the user to feel ill effects from the inversion.

3. Identification of Objects to the Invention

An object of the invention is to accomplish one or more of the following:

Provide a machine which vertically stretches the human frame by partially or fully suspending a user from the user's upper body without the ill effects of inversion;

Provide a machine which vertically stretches the human frame by partially or fully suspending a user from the user's upper body up to forty degrees relative to the horizon; and

Provide a machine which permits the user to precisely control the degree of vertical stretching which occurs by controlling the degree to which the user's upper body is partially or fully suspended.

Other objects, features, and advantages of the invention will be apparent to one skilled in the art from the following specifications and drawings.

### SUMMARY OF THE INVENTION

An exercise machine and method are disclosed for stretching and exercising the human body through vertical suspension. The machine preferably has a bed, table, or platform upon which the user lies and becomes physically attached thereto while using and operating the machine. The user's upper body is restrained to the table using a jacket vest or similar apparatus with an attachment mechanism capable of supporting the user's entire weight while the table is in a non-horizontal position. The machine preferably has a user-controlled actuator which raises and lowers the user's upper body within a forty degree range above a completely horizontal position. In this way, the user's upper body is raised above the user's lower body. As a consequence, the user's lower body is either partially or fully suspended from the user's point of attachment to the table.

In a preferred method, the user lies on the bed in a supine position using the jacket vest or similar apparatus to physically attach the upper body to the table. The user then controls the self-actuator to raise (and/or lower) one end of the table so that the user's upper body is raised upwardly between zero to forty degrees while the user's lower body positioned at the opposite end of the table is lowered by the same degree. Thus, the table is arranged and designed to pivot about the support frame while the actuator is lifting the table. Because the user controls the amount and duration of vertical suspension using the precise actuator movements, the machine is much easier and safer to operate and more effective in vertically stretching and exercising the human frame than other exercise and physical strengthening devices.

### BRIEF DESCRIPTION OF THE DRAWINGS

The objects, advantages, and features of the invention will become more apparent by reference to the drawings which are appended hereto, and wherein by way of illustration and not limitation implementations of the inventions are described, of which:

FIG. 1 illustrates a preferred implementation of the machine for stretching and exercising the human body through vertical suspension wherein the table, atop which the user lies while operating the machine, is in the starting or horizontal position; and

FIG. 2 illustrates a preferred implementation of the machine for stretching and exercising the human body through vertical suspension wherein one end of the table is elevated above its frame by a user-controlled actuator so that the user's upper body, which is physically attached to the table, is higher than the user's lower body, thus partially or fully suspending the user's lower body.

### DESCRIPTION OF THE PREFERRED EMBODIMENTS OF THE INVENTION

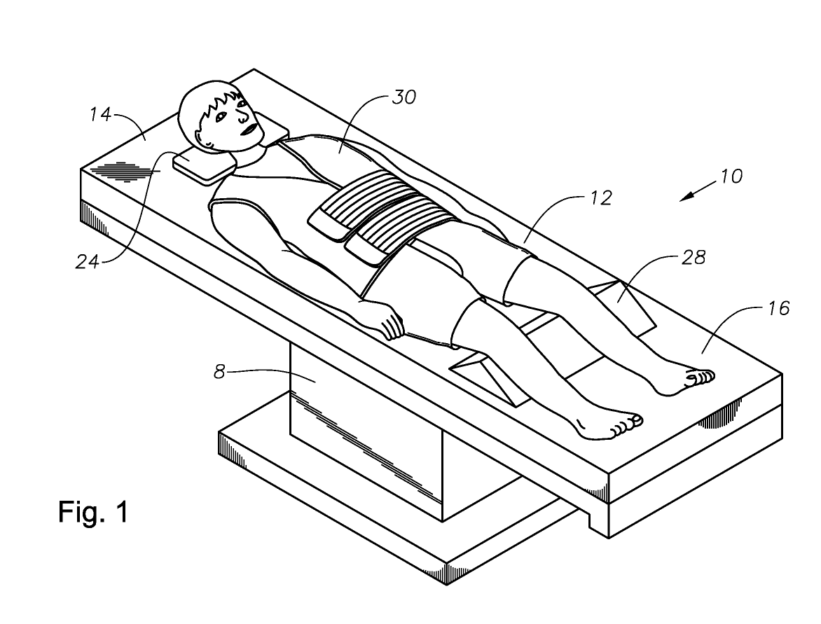

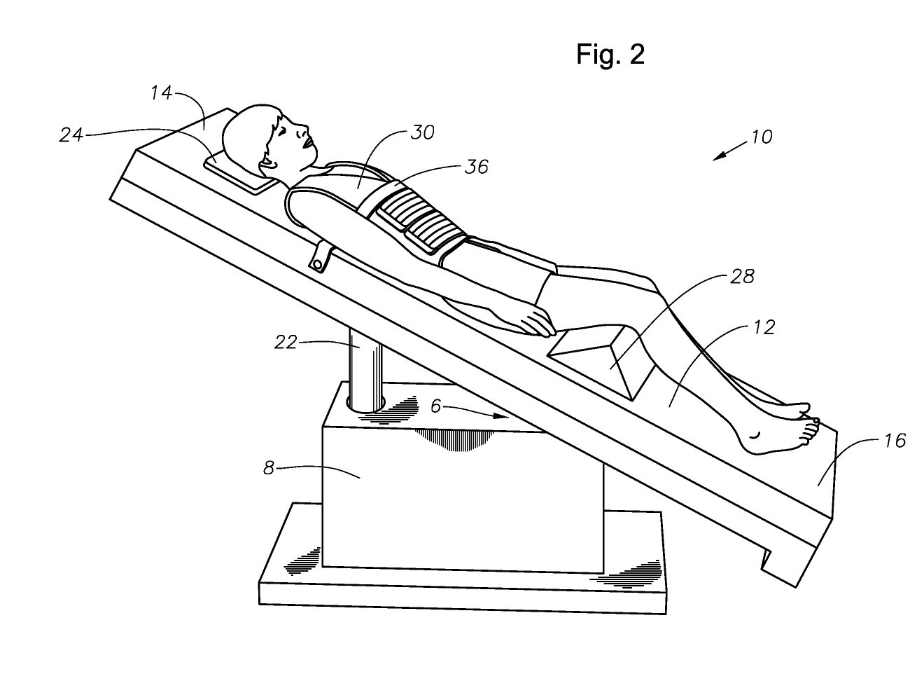

As generally shown in FIG. 1, a preferred implementation of the invention is a machine 10 for stretching and exercising the human body through vertical suspension. The machine 10 has a bed, table, or platform 12 upon which the user is positioned during operation. The table 12 is comprised of two main sections: (1) an upper section 14 upon which the user's upper body is physically attached and (2) a lower section 16 which receives the user's lower body. The table 12 is preferably disposed a comfortable distance above ground level using a frame 8, which is capable of fully supporting the user's full weight. The table 12, while being supported by the frame 8, is not fully secured thereto. As shown in FIG. 2, an actuator 22 is arranged and designed to raise the upper section 14 of the table 12 up to forty degrees above the frame 8. The lower section 16 of the table 12 is attached to the frame 8 in such a way as to pivot about the frame 8 while the actuator 20 is lifting or lowering the upper section 14 of table 12.

As shown in FIGS. 1 and 2, the user's upper body is restrained to the upper section 14 of the table 12 using a jacket vest 30 or similar apparatus with an attachment mechanism capable of supporting the user's entire weight while the table 12 is in a non-horizontal position. Hook and loop fasteners, such as Velcro®, are the preferred attachment mechanism for attaching the jacket vest 30 to the table 12, however, other attachment mechanisms are equally suitable. A matrix of hook or loop fasteners is preferably disposed on the back of the jacket vest 30 while the corresponding matrix of hook or loop fasteners is disposed on top of the upper section 14 of the table 12. The matrix of hook or loop fasteners on the back of the jacket vest 30 permits the wearer to position the body in a supine position atop the table 12. When properly engaged, the hook and loop fasteners of a preferred implementation can support approximately 1,200 pounds. However, a safety cord or similar safety device 36, which is connected at both ends to the table 12 and fits underneath the user's arms (i.e., between the user's arms and trunk) and across the user's chest, is preferably employed in addition to the hook and loop fasteners to ensure that the user does not become fully detached from the table 12 during machine operation.

In a preferred implementation of the machine 10, a knee wedge 28 is positionally adjusted atop the lower section 16 of the table 12 for placement of a user's legs thereover while the user is positioned on the table 12. The knee wedge 28 changes the angle of the user's pelvis thereby creating a more comfortable position for the user which is similar to sitting. The knee wedge 28 remains physically attached to the table 12 while the machine 10 is in operation. Optionally, a head rest 24 or head cushion may be positioned on the upper section 14 of the table 12 to cradle the user's head, neck, and upper shoulders in the supine position while using the machine 10.

As previously described, an actuator 22 is arranged and designed to raise and lower the upper section 14 of the table 12 within a range of up to forty degrees above the attached frame 8. The lower section 16 of the table 12 is attached to the frame 8 in such a way as to pivot about the frame 8 while the actuator 22 is lifting or lowering the upper section 14 of the table 12. Actuators and other powered mechanisms which move and manipulate physical objects relative to each other are well known in the art and thus will not be discussed at length herein. Likewise, control interfaces for actuators and similar mechanisms, which are operable by the user, are also well known in the art and include, but are not limited to, remote control devices, hard wired devices, wireless devices and voice command systems. The actuator 22 in a preferred implementation of the machine 10 is user-controlled so that the user may raise or lower the user's upper torso to a level of greatest comfort over a forty degree range relative to the horizon. The user-actuated position control (not shown) also permits the user to vary on demand the vertical distance the table 12 is raised or lowered as well as the duration the table 12 remains at any particular position. For example, the user may choose to position themselves at 10-15-20 or even 30 degrees (as shown in FIG. 2) for 3-4 minutes, and then return to the horizontal starting position (as shown in FIG. 1). Similarly, the user may raise and then lower the table 12 progressively before achieving a maximum of 30 or 40 degrees.

In a preferred method, the user lies on the table 12 in an supine position using the jacket vest 30 or similar apparatus to physically attach the upper body to the upper section 14 of the table 12. The user then uses the self-actuator control (not shown) to raise (and subsequently lower) the upper section 14 of table 12 so that the user's upper body is effectively raised above the horizon by as much as forty degrees. Because the user's upper torso is physically attached to the table 12 through the jacket vest 30, the user's lower body actually becomes either partially or fully suspended depending on the degree the table 12 is raised. The combination of the jacket vest 30 and the knee wedge 28 allows gravity to pull on the user's upper and lower body thereby stretching and unloading the human frame. As pressure is reduced on the lower body, the user becomes relaxed and experiences an increased sense of well being. Because the user controls the amount and duration of vertical suspension using the precise actuator movements, the machine 10 permits the user to accomplish the degree of vertical stretching and exercising that is most comfortable. In this way, the machine 10 accommodates a wide variety of human morphologies while being much easier and safer to operate than other exercise and physical strengthening devices.

The Abstract of the disclosure is written solely for providing the United States Patent and Trademark Office and the public at large with a means by which to determine quickly from a cursory inspection the nature and gist of the technical disclosure, and it represents one implementation of the invention and is not indicative of the nature of the invention as a whole.

While some embodiments of the invention have been illustrated in detail, the invention is not limited to the embodiments shown; modifications and adaptations of the above embodiments may occur to those skilled in the art. Such modifications and adaptations are in the spirit and scope of the invention as set forth herein:

What is claimed is:

1. An exercise machine (10) for stretching a human body comprising,
a support frame (8) designed to support a table above ground level,
a table (12) arranged and designed to support a user in a supine position, said table having an upper section (14) which receives a user's upper body and a lower section (16) which receives a user's lower body, said table moveably disposed atop said support frame,
an actuator (22) connected to said table and said support frame, said actuator arranged and designed to raise said upper section of said table above said support frame while said lower section of said table pivots about an end (6) of said support frame, and
a mechanism for preventing longitudinal movement of said user's upper body about said upper section of said table, said mechanism comprising a jacket (30) having an attachment device arranged and designed to cooperatively fasten to said upper section of said table, said attachment device comprising a hook and loop fastener.

2. The machine of claim 1 wherein,
said actuator is controlled by a user of said machine.

3. The machine of claim 2 further comprising,
a safety strap (36) having two ends, said safety strap arranged and designed to be disposed across a user's chest and between a user's arms and trunk and to be connected to said table at both of said ends.

4. The machine of claim 3 further comprising,
a knee wedge (28) disposed on said lower section of said table for placement of a user's legs thereover while a user is supported by said table.

5. The machine of claim 1 wherein,
said actuator is arranged and designed to raise said upper section of said table to between ten and twenty degrees above said support frame.

6. The machine of claim 1 wherein,
said actuator is arranged and designed to raise said upper section of said table to between twenty and thirty degrees above said support frame.

7. The machine of claim 1 wherein,
said actuator is arranged and designed to raise said upper section of said table to between thirty and forty degrees above said support frame.

8. The machine of claim 1 further comprising,
a safety strap (36) having two ends, said safety strap arranged and designed to be disposed across a user's chest and between a user's arms and trunk and to be connected to said table at both of said ends.

9. The machine of claim 1 further comprising,
a knee wedge (28) disposed on said lower section of said table for placement of a user's legs thereover while a user is supported by said table.

10. An exercise machine (10) for stretching a human body comprising,
a support frame (8) designed to support a table above ground level,
a table (12) arranged and designed to support a user in a supine position, said table having an upper section (14) which receives a user's upper body and a lower section (16) which receives a user's lower body, said table moveably disposed atop said support frame,
an actuator (22) connected to said table and said support frame, said actuator arranged and designed to be controlled by a user and to raise said upper section of said table above said support frame while said lower section of said table pivots about an end (6) of said support frame,
a mechanism for preventing longitudinal movement of said user's upper body about said upper section of said table, and
a safety strap (36) having two ends, said safety strap arranged and designed to be disposed across a user's chest and between a user's arms and trunk and to be connected to said table at both of said ends.

11. The machine of claim 10 further comprising,
a knee wedge (28) disposed on said lower section of said table for placement of a user's legs thereover while a user is supported by said table.

12. The machine of claim 10 wherein,
said actuator is arranged and designed to raise said upper section of said table to between ten and twenty degrees above said support frame.

13. The machine of claim 10 wherein,
said actuator is arranged and designed to raise said upper section of said table to between twenty and thirty degrees above said support frame.

14. The machine of claim 10 wherein,
said actuator is arranged and designed to raise said upper section of said table to between thirty and forty degrees above said support frame.

15. An exercise method for stretching a human body comprising the steps of,
positioning a user on a table (12) arranged and designed to support said user in a supine position, said table having an upper section (14) which receives a user's upper body and a lower section (16) which receives a user's lower body, said table being supported above ground level by a support frame (8),
employing a mechanism to prevent a user's upper body from longitudinally moving about said upper section of said table, said mechanism comprising a jacket (30) having an attachment device arranged and designed to cooperatively fasten to said upper section of said table, said attachment device comprising a hook and loop fastener,
operating an actuator (22) connected to said table and said support frame to raise said upper section of said table above said support frame while said lower section of said table pivots about an end (6) of said support frame, said actuator arranged and designed to be controlled by said user while said user is supported by said table.

16. The method of claim 15 further comprising the step of,
employing a safety strap (36) across a user's chest and between a user's arms and trunk to prevent longitudinal movement of said user about said upper section of said table when said actuator is being operated.

17. The method of claim 16 further comprising the step of,
positioning a user's legs over a knee wedge (28) disposed on said lower section of said table.

18. The method of claim 16 wherein,
said actuator is arranged and designed to raise said upper section of said table to between ten and forty degrees above said support frame,

19. The method of claim 15 further comprising the step of,
positioning a user's legs over a knee wedge (28) disposed on said lower section of said table.

20. The method of claim 19 wherein,
said actuator is arranged and designed to raise said upper section of said table to between ten and forty degrees above said support frame.
